(19) United States
(12) Patent Application Publication
(10) Pub. No.: US 2015/0210975 A1
(43) Pub. Date: Jul. 30, 2015

(54) SYSTEMS AND METHODS FOR A CONTINUOUS CULTURE BIOSENSOR

(71) Applicants: Holly Vee Goodson, South Bend, IN (US); Julia Theresa Ott Philip, South Bend, IN (US)

(72) Inventors: Holly Vee Goodson, South Bend, IN (US); Julia Theresa Ott Philip, South Bend, IN (US)

(73) Assignee: University of Notre Dame du Lac, Notre Dame, IN (US)

(21) Appl. No.: 14/682,828

(22) Filed: Apr. 9, 2015

Related U.S. Application Data
(62) Division of application No. 13/423,801, filed on Mar. 19, 2012, now abandoned.

(51) Int. Cl. C12M 1/36

(52) U.S. Cl. CPC C12M 41/48

(57) ABSTRACT

A continuous culture system of genetically modified yeast or another biological organism detects contaminants in water through production of a fluorescent chemical continuously produced by the organism after it is contacted with a threshold concentration of a contaminant. Alternatively, a biological organism can detect toxins or extra nutrients by detecting a change in growth rate. Biological organisms can also be measured for mutagenic changes by comparing their genome with a control sample. A network of continuous culture systems may be used as part of a water contamination detection system for real-time water monitoring of contaminants from multiple sources simultaneously.

### CROSS REFERENCE TO RELATED APPLICATION

This application is a divisional of U.S. patent application Ser. No. 13/423,801, filed Mar. 19, 2012, which claims priority to U.S. Provisional Patent Application No. 61/465,335 entitled “A Yeast-Based Biosensor for Continuous Monitoring of Water Quality” filed Mar. 17, 2011, and U.S. Provisional Application No. 61/630,010 entitled “A Yeast-Based Biosensor for Continuous Monitoring of Water Quality” filed Dec. 2, 2011, each of which is incorporated herein by reference in its entirety.

### FIELD OF THE DISCLOSURE

The present disclosure relates generally to biosensors and more particularly to systems and methods for a continuous culture biosensor.

### BACKGROUND

In the monitoring of water quality, continuous testing can be expensive, and there can be a significant time delay between when a pollutant becomes introduced into the water and its subsequent detection.

Water quality is a critical part of environmental health. Due to the crucial nature of water monitoring, there are many products for continuous, in situ water quality detection. However, existing technologies chiefly monitor general water quality parameters such as total organic carbon and pH rather than specific water contaminants. While monitoring these parameters can detect water contamination events, it is difficult to identify specific contaminants. Technologies to detect specific contaminants generally require either sample collection for laboratory analysis or a technician using a hand-held testing device at the water location. These monitoring solutions are typically costly, both in terms of equipment and trained personnel to operate and maintain the sensor systems, limiting water network monitoring.

The majority of current technologies for water quality testing rely on collecting water samples and then testing them in a laboratory setting. This process is oftentimes time consuming and requires highly trained personnel. The few portable sensors on the market require both operation by trained personnel and frequent calibration. As a result, many at-risk bodies of water are rarely, if ever, tested. Current water quality testing technology also causes problems for municipal wastewater treatment systems, which are charged with monitoring local industries for contaminant dumping. They rely on testing water as it enters the treatment plant, where any contamination will have been greatly diluted and sources are hard to track.

Known existing in-line continuous monitors for wastewater are very expensive and maintenance-intensive, and this high cost restricts the number of devices that can be deployed and the fraction of the network that can be monitored. Furthermore, the known existing continuous monitors are limited in the number of parameters they can test, with characteristics typically limited to nonspecific characteristics including residual chlorine, total organic carbon, oxidation-reduction potential, pH, dissolved oxygen, conductivity, turbidity, and temperature. This limitation allows many toxins to pass unnoticed.

Municipal wastewater treatment facilities utilize biological processes (microbes) to treat waste. While these microbes are relatively robust, as living organisms, they are susceptible to toxins that are released on occasion (intentionally or unintentionally) into the sewers, damaging the plant and potentially putting it off-line for months. Such events are catastrophic both economically (due to fines and restart costs) and ecologically (through the release of the original toxin and subsequent untreated sewage).

Municipal wastewater treatment facilities are responsible for monitoring industry effluent. Many types of testing are only performed at specific intervals, allowing noncompliant events to be missed. Moreover, effluent is typically tested after water from many sources is mixed, rendering it diluted and difficult to detect the ultimate source when inappropriate contaminants are detected.

Eutrophication of surface water (pollution by oversupply of nutrients) results from over-fertilization of lawns and crops, and it also comes from farm effluent, particularly confined animal operations. Eutrophication causes algal blooms, damaging ecosystems and resulting (eventually) in problems such as the dead zone at the mouth of the Mississippi or the algal blooms impacting Lake Erie. As there are many potential sources of eutrophication of surface water, it is often expensive and time consuming to determine the offending source such that corrective measure can be taken.

Thus, conventional technology has not provided a successful and cost-effective means by which to continuously monitor certain contaminants, from a wide range of sources. A device that could be placed near a water source for continuous long term monitoring would be a useful tool to ensure compliance with applicable regulations and to timely detect and rectify water quality violations.

### BRIEF DESCRIPTION OF THE DRAWINGS

FIG. 1 is a diagram of genetically modified yeast genes that create a memory system that emits a green fluorescent protein after coming into contact with the sugar galactose.

FIG. 2 is a picture of plates containing yeast cultures under a light source, some of which have been genetically modified, on media which contains galactose and glucose.

FIG. 3 is a picture of yeast colonies on a plate under a light source, some of which have been genetically modified.

FIG. 4 is a diagram of an example detector device.

FIG. 5 is a picture of an example bench scale detector device, with a 250 ml yeast chamber.

FIG. 6 is a picture of a 5 ml yeast chamber, as part of another example bench scale detector device.

FIG. 7 is a graph depicting yeast culture life in a 5 ml yeast chamber bench scale detection device, by measuring conductivity while the yeast culture is alive, and after a toxin is introduced to kill the yeast.

FIG. 8 is a graph showing the correlation of conductivity readings to photospectrometer readings for four strains of yeast cultures.

FIG. 9 is a graph demonstrating the response of a fluorescence detector to fluorescent beads.

FIG. 10 is an image of a six well plate in the in vivo imager containing yeast cultures and beads.

FIG. 11 is a diagram of linear homologous recombination to replace only the promoter in genetically modified yeast genes.

FIG. 12 is a diagram of homologous recombination using a yeast integrating plasmid to replace the promoter and add nutritional selection in genetically modified yeast genes.

FIG. 13 is a picture of genetically modified yeast colonies plated on media with and without copper.

### DETAILED DESCRIPTION

Reference will now be made in detail to various examples of the present disclosure, which are illustrated in the accompanying drawings. While the present disclosure will be described in conjunction with various examples, it will be understood that the disclosure is not intended to limited to these examples. On the contrary, the present disclosure is intended to cover alternatives, modifications and equivalents, which may be included within the spirit and scope of the disclosure as defined by the claims. Furthermore, in the detailed description of the present disclosure, numerous specific details are set forth in order to provide a thorough understanding of the teachings of the present invention. However, it will be obvious to one of ordinary skill in the art that the present invention may be practiced without these specific details. In other instances, well known methods, procedures, and components have not been described in detail as not to unnecessarily obscure aspects of the present invention.

Generally speaking, examples provide for methods for monitoring a threshold concentration of a particular chemical in an aqueous medium through the use of a continuous culture system. In particular, examples provide for methods for preparing, using, and monitoring continuous culture systems. While the specification makes reference to the culture as a yeast, another biological organism culture may be substituted such as bacteria or virus, in which known genetic markers are added which turn on, and sustain, the fluorescence emitter when in contact with a threshold level of a chemical. In certain examples of the present disclosure, the biological organism may not be genetically modified at all. Furthermore, in additional examples, the present disclosure may be used in salt water, for which yeast may not be the most suitable organism. Yeast may be utilized over bacteria and virus, because yeast are more tolerant of variations in environmental conditions (temperature, salt, etc.) and because the extensive yeast genetic engineering toolbox allows for straightforward creation of specialized sensing strains.

The continuous culture system is composed of two components: a) a living yeast strain, and b) a detector device, which will house the yeast, deliver test water and growth media to them, query them for signal indicating contaminant exposure, growth or the lack thereof, and send signals to a monitoring station. In one example of the present disclosure, the yeast is genetically engineered to respond to a specific contaminant or class of contaminants. The genetically engineered yeast will remember previous exposure to contamination, thus producing a detectable signal in response to both transient and sustained water contamination. Such a system may function independently for one to six months and should not require advanced training to operate or service.

Genetically engineered yeast sensor strains function as a toggle switch, changing from off to on at a defined contaminant concentration. This system functions as an alarm that can trigger a more thorough investigation of the water quality. Individual sensor devices are relatively inexpensive and easy to maintain. In addition, this system allows a level of continuous monitoring of water streams that is impractical with existing technologies. An additional benefit of this toggle switch system is that avoids reduced expression in the event of chronic contaminant exposure, which has been shown to reduce expression of toxin-responsive genes.

Referring now to the drawings, yeast strains have been genetically modified from common bakers' yeast Saccharomyces cerevisiae as described by Dr. Pam Silver (Ajo-Franklin C M, Drubin D A, Eskin J A, et al. Rational design of memory in eukaryotic cells. Gene Dev. 2007; 21(18):2271-2276), in conjunction with a memory system, wherein the exposure of a genetically modified yeast to the sugar galactose triggers the genetic cascade described below and in FIG. 1.

Exposure of the yeast to galactose causes an endogenous galactose binding protein 14 to bind to and drive expression from the Gal1 promoter 18, present in a strain of genetically modified yeast. This causes production of a protein 14. The protein 14 then binds to and drives expression from the Cycl promoter 26, also present in the strain of genetically modified yeast. This causes production of a protein 16. The protein 16 has two important characteristics: first, it contains a green fluorescent protein (GFP) 28; and second, it also contains a DNA binding domain 30 that stimulates its own production, creating a positive feedback loop. Thus once exposed to galactose, this particular genetically modified yeast strain will continuously produce GFP 28 even after the galactose is removed. Both of these genetic elements 10 and 12 are integrated into the yeast chromosome, which eliminates any variability due to copy number variation or loss of the DNA.

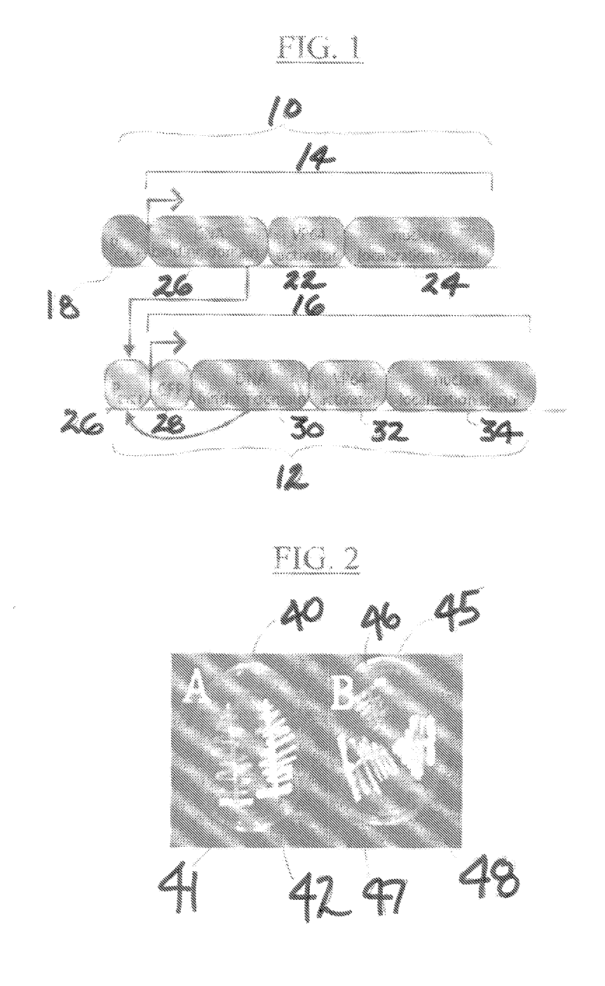

The Gal1 promoter used in the memory strain described (and provided) by Dr. Silver has behaved as expected in responding to galactose. As shown in FIG. 2, strains of yeast are placed on two plates: plate 40 contains galactose, while plate 45 does not. Yeast strain 41 is a control strain that does not contain the memory system described above, and merely reflects light. Yeast strain 42 contains the memory system described above, and both reflects light and emits a green fluorescent signal. Plate 45 contains media containing glucose, and no galactose. Yeast strain 46 is a control strain that does not contain the memory system as described above, and only reflects light. Yeast strain 47 contains the memory system described above, but has only been exposed to glucose. It, too, only reflects light. Yeast strain 48 contains the memory system described above, and was previously plated with galactose, and was found to continuously emit a green fluorescent signal for at least two days after the galactose is no longer present.

Based on this strain, new strains may be constructed that respond to water contaminants. The genetic modification necessary to generate yeast strains that respond to different triggers depends on replacing the pGal1 promoter with a promoter that responds to the contaminant of interest. This is accomplished by using standard yeast transformation techniques. Briefly, a piece of DNA containing the new promoter flanked on both sides by regions identical to the DNA in the existing strain is introduced into the yeast. The yeast then performs homologous recombination, which replaces the promoter in the initial strain with the new promoter. Protocols for this procedure are well established and employed in many laboratories.

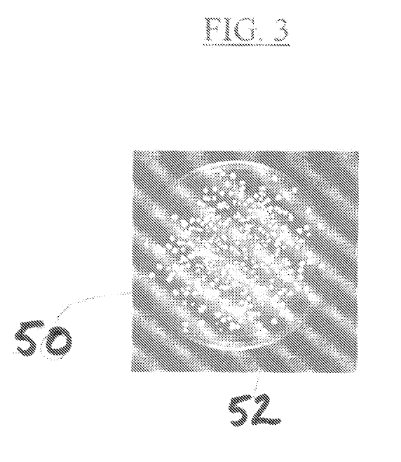

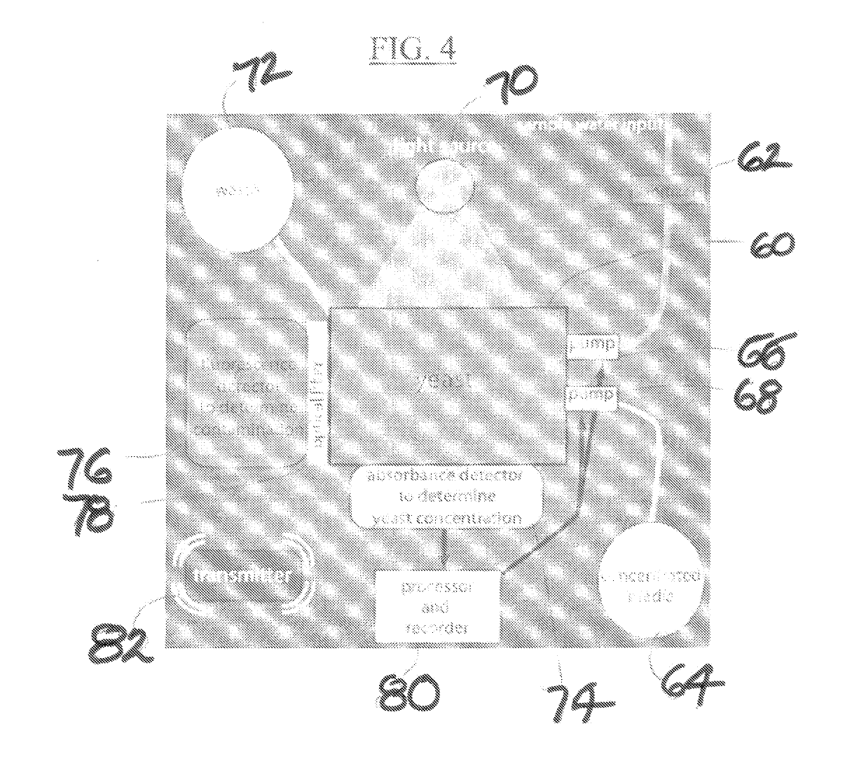

The yeast is transformed with both the DNA to introduce the new promoter and a plasmid with genes to rescue a nutritional deficiency at the same time. It has been shown that cotransformation is a viable method for introducing two pieces of DNA into yeast simultaneously. After transformation, yeast is selected that have taken up the plasmid using appropriate restrictive media. Yeast is then screened, to only include yeast that have properly incorporated the new promoter by replica-plating the yeast onto a plate containing the contaminant that the new promoter responds to, and then imaging the plates to identify fluorescent yeast colonies. Using this approach, thousands to tens of thousands of yeast colonies may be screened in a less than an hour. FIG. 3 demonstrates the transformant screening procedure. A galactose-containing plate 50 contains single yeast colonies 52. The yeast colonies that only contain the control strain, and not the memory strain, only emit a gray light as a reflection from a light source. The yeast colonies that contain the memory strain reflect a green fluorescent light in response to the light source. Responsive yeast strains are then tested to establish their contaminant response threshold.

The estrogen response promoter responds to human estrogen. Environmentally problematic levels of estrogen and estrogenic compounds generally will trigger the estrogen response promoter. When imported into yeast, this system has been shown to respond to estrogen in environmental samples. For this strain the Gal1 promoter is replaced with an estrogen responsive promoter. The human estrogen binding protein is used, which turns on protein expression at the estrogen responsive promoter after binding estrogen, into the yeast strain. This strain imports a toxin response system from another organism.

The Cup1 promoter is native to yeast and has been used successfully as a mechanism of controlling protein expression in yeast. This promoter has a relatively high level of expression in the absence of copper, which may cause it to trigger in the absence of stimulus. The Cup1 promoter was chosen to overcome this problem that may occur with some other attractive response systems; studies with this promoter test techniques such as using degradation tags to shorten the lifetimes of the initial protein and mRNA to overcome this problem.

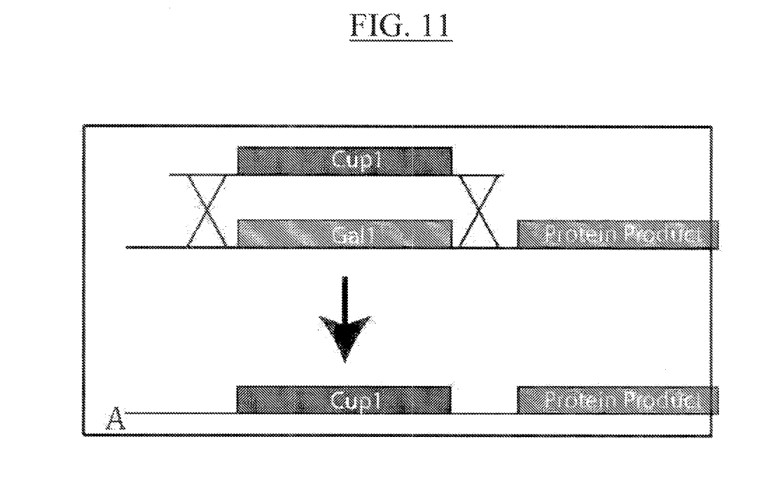

Genetic replacement relies on the yeast's ability to perform homogenous recombination to replace the Gal1 promoter with the Cup1 promoter. FIG. 11 is a diagram of linear homologous recombination to replace only the promoter. The yeast was cotransformed with the linear DNA for promoter replacement and a plasmid with nutritional selection. After selecting for the yeast that took up the plasmid, the colonies were screened for the promoter replacement. However, this method was found to be impractical, as promoter replacement occurs only once in every 500 to 1000 colonies. Screening for such a rare event presents a significant technical challenge.

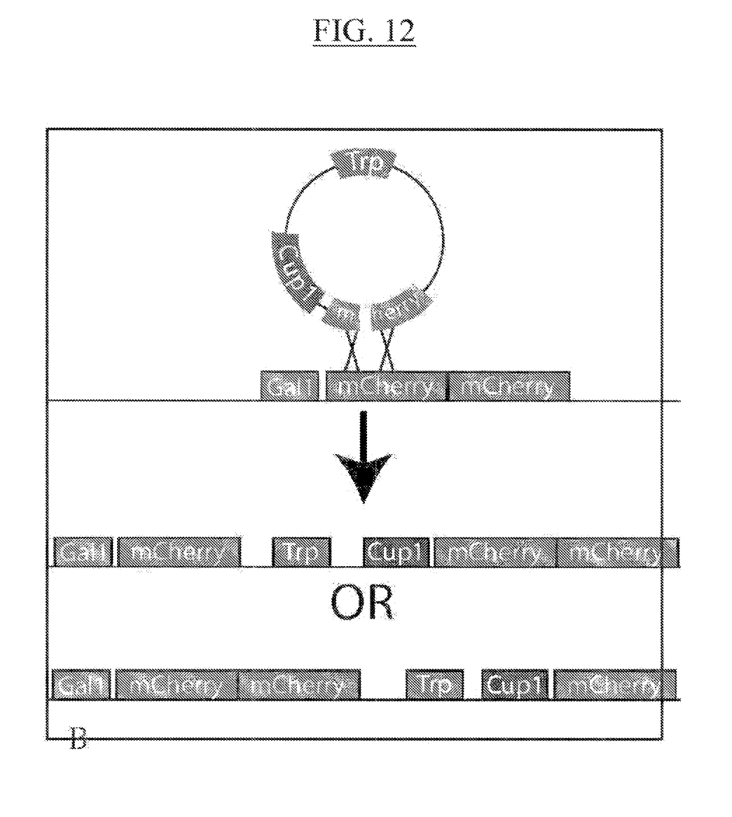

To overcome this challenge, a yeast integrating plasmid is used. FIG. 12 is a diagram of homologous recombination using a yeast integrating plasmid to replace the promoter and add nutritional selection. In this technique, yeast's ability to carry out homologous recombination is exploited by creating a plasmid containing a region with homology to the yeast chromosome. Prior to transformation this plasmid is restriction enzyme digested in the homologous region to linearize the plasmid. After transformation the yeast will integrate the entire plasmid into their chromosomes. In addition to the new promoter, the plasmid carries a nutritional selection region, allowing easier selection of the yeast that integrated the plasmid.

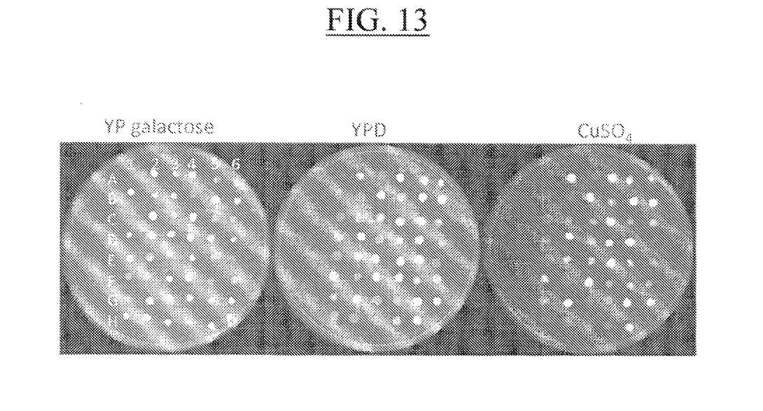

FIG. 13 shows transformed colonies plated on media with and without copper, demonstrating a success rate of greater than 50%. FIG. 13 shows plates of colonies of transformants imaged in the in vivo imager for YFP. Colony A1 is a control yeast with no fluorescence. Colony B1 is the original memory strain. Many transformed colonies show response to copper, for example colony B6. Proper plasmid integration was confirmed with PCR.

Three yeast genes that are not expressed under normal conditions and are highly expressed in response to arsenic are YKL071W, YPL171C and YCL026C-A. Promoters for these genes are used to construct arsenic sensing strains. These promoters are desirable because of their utility as sensors (arsenic contamination is a problem in both the developed and developing world) and because of the tightly regulated nature of these promoters. Yeast promoters that respond to chemical stress may also be used.

To import contaminant sensing systems from other organisms, knowledge of both the identity of the response promoter and the contaminant binding protein are needed to induce the yeast to produce the contaminant binding protein. In searching for genetic response systems for contaminants of interest, generally it is helpful to search for bacteria or other microorganisms that are tolerant to high concentrations of the contaminant. These organisms will generally have genetic systems to detect and respond to the compound of interest. Organisms with well characterized contaminant response systems provide such a source of knowledge. In particular, Ralstonia sp. CH34 is a good source of metal responsive systems, as it has been shown to respond to cobalt, zinc, cadmium, mercury, chromate, and nickel. Alternatively, mutagenesis and directed evolution approaches may also generate the desired organism.

In one example, a detector device houses the yeast to allow the yeast to be in continuous contact with water to be tested for contamination. A schematic of an example device 59 is shown in FIG. 2. The components of the example device 59 include a yeast chamber 60, a concentrated media 64, a sanitizer 62, a light source 70, photodetectors 74 and 76, an optical filter 78, pumps 66 and 68, a waste outlet 72, a processor 80, and a transmitter 82. FIG. 2 is a schematic, and is not meant to refer to any relative sizes or positions of the equipment described therein; it is merely meant to describe the components' relationship with one another. It should be noted that this is just one example of the present disclosure, and is not meant to limit the claimed invention to the precise combination disclosed.

In operation, the water to be tested is drawn in through a sanitizer 62 by a pump 66 and added to a yeast chamber 60. In this example, the yeast chamber 60 is transparent and/or translucent, so the light used to periodically query the yeast, and the photodetectors used to measure the response, will not be obscured. Concentrated media 64 includes nutrients used to promote yeast growth and is added by pump 68 as needed. Water flows out of waste outlet 72. A blue light emanating from light source 70 periodically queries the yeast. Two photodetectors measure the response: photodetector 74 measures absorbance to determine the yeast concentration and photodetector 76 detects the presence or absence of the fluorescent signal indicating the presence of the compound of interest. Optical filter 78 assists photodetector 76 in isolating the particular green light emitted by GFP to be detected. Processor 80 receives data from both photodetectors 74 and 76, and control the flow of water to be tested, and concentrated media to maintain the yeast concentration at an appropriate level, through control of pumps 66 and 68, as well as to trigger an alarm when necessary using transmitter 82 to a centralized computer or location.

Sanitizer 62 is used to prevent foreign microorganisms in the intake water from taking over the yeast culture, destroying the response of the sensor. While the present example describes the use of sanitizer 62, it will be appreciated by one of ordinary skill in the art that the example detector device may utilize any alternative or additional means for preventing catastrophic death of the yeast culture, such as for example filtering or settling, or adding a bacterial toxin to the yeast media.

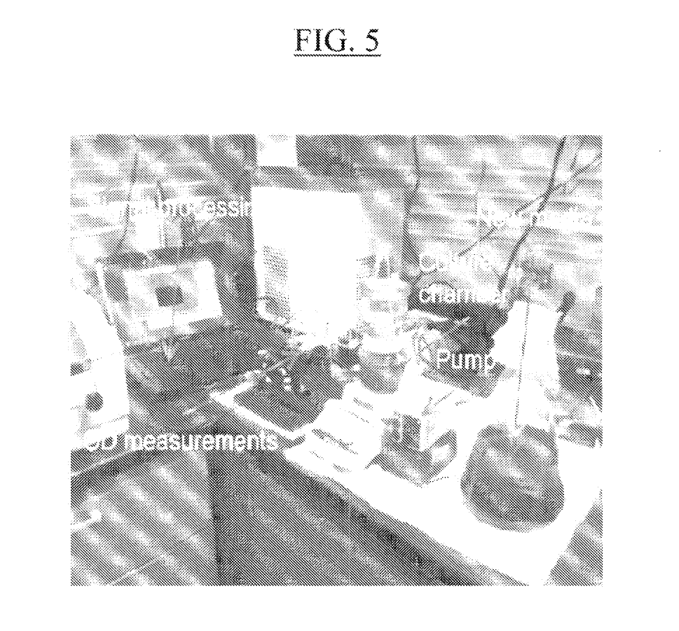

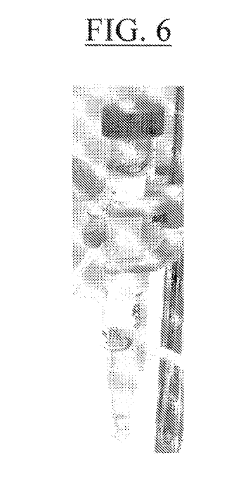

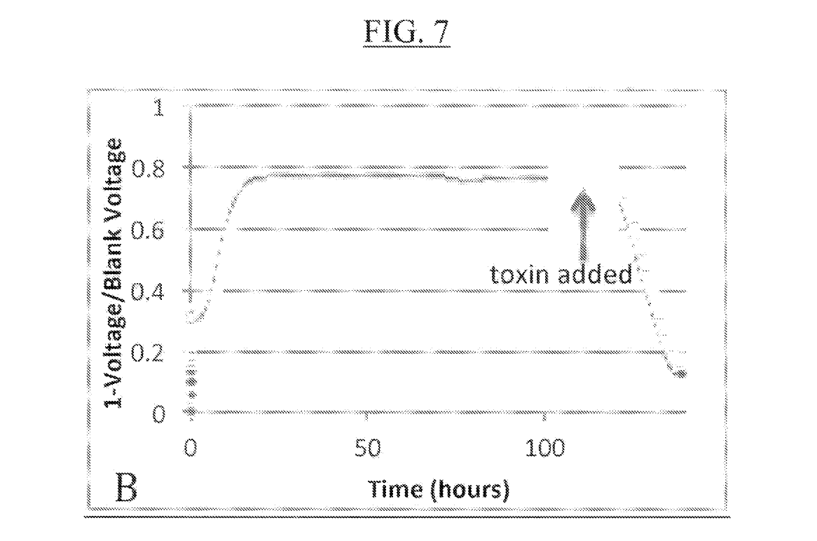

Examples of the detector device described above are illustrated in FIGS. 5-6, which are laboratory versions. FIG. 5 utilizes a 250 ml culture chamber, while FIG. 6 utilizes a 5 ml culture chamber. Both devices have operated for months at a time. The 5 ml chamber is more practical for long-term remote use because of its minimal use of concentrated media. FIG. 7 is a conductivity graph that demonstrates the presence of a yeast culture of the 5 ml chamber of FIG. 6 over a period of 115 hours, at which point a toxin was added, and the subsequent catastrophic yeast death was recorded.

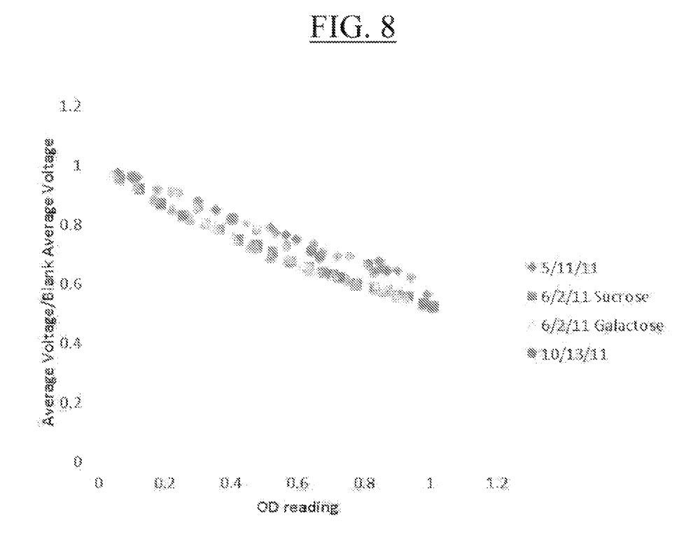

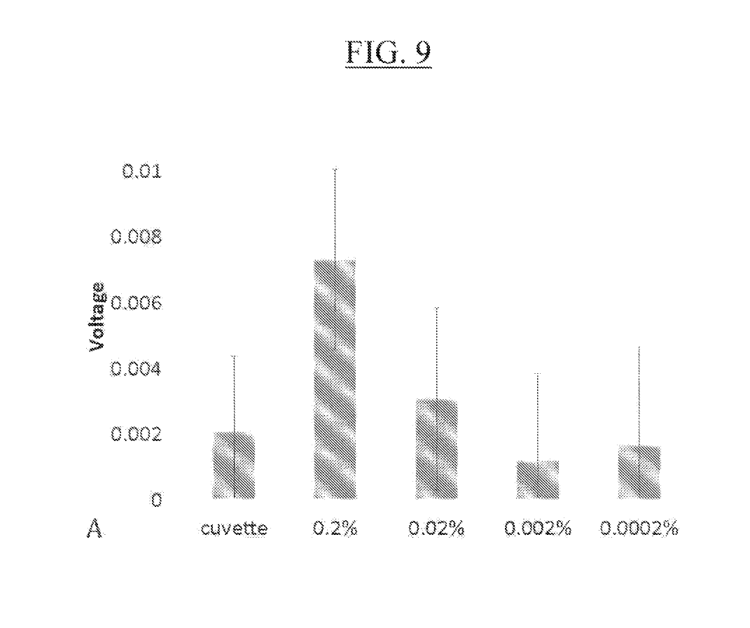

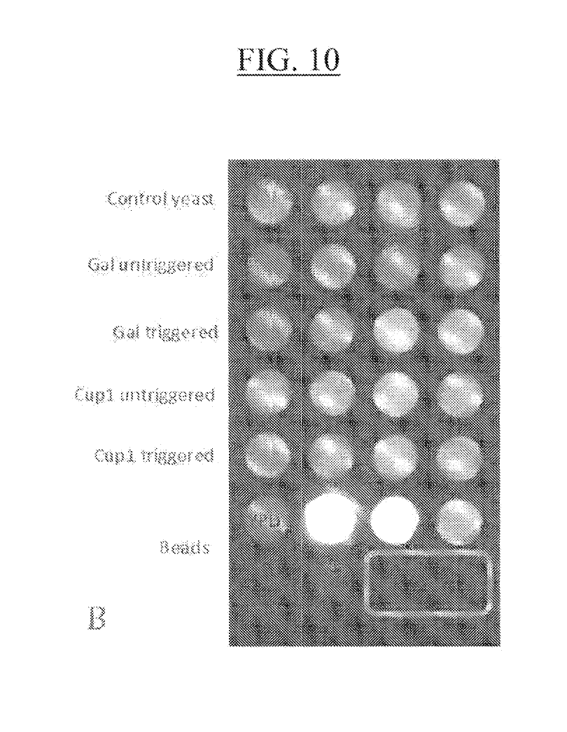

Cell density is then measured by detecting the amount of light from an LED that travels through a cuvette containing the culture to a photodiode detector. In one instance, the disclosed measuring method is similar to cell density measurement using a photospectrometer. FIG. 8 shows a comparison of cell density measurements from a photospectrometer and the present device for a wide range of culture densities. FIG. 8 shows OD readings from a photospectrometer compared to normalized voltage readings for four different yeast cultures. A line to fit to the combined data from all four cultures has an R² value of 0.92.

The detector device can be configured to contain multiple independent chambers holding different yeast strains, each detecting a different contaminant. While individual photodetectors will be needed for each yeast chamber, such a system may require only one light source, and the pumps may be used to deliver water and concentrated media to all of the chambers. Furthermore, each independent chamber located in the same detector device may be connected to the same processor and transmitter. Such a device will enable a faster response to water contamination as the response of particular strains will indicate which contaminant is present. In addition, the device will be designed to operate either using the standard power grid or using a battery. This will allow these devices to be deployed for long term water monitoring in remote locations.

In another example of the present disclosure, the yeast chamber is periodically evacuated to test the yeast in a separate chamber that may enable easier or more cost effective testing, and then the yeast culture returned to the yeast chamber.

In addition to estrogen and copper, this present device may also be used to detect a wide range of contaminants in this manner, including arsenic, cobalt, zinc, cadmium, mercury, chromate, nickel, other metals, pharmaceuticals, endrocrine disruptors, fertilizers, pesticides, cleaning agents, industrial wastes, fracking chemicals, and petrochemicals.

The example detector device may, in one example, be used in a water monitoring and treatment system as desired. In particular, in one example, a detector device constructed in accordance with the present disclosure can be combined with others into a network of sensors as an inexpensive way to monitor entire wastewater streams to quickly detect any improper release of contaminants. The detector devices may be designed to utilize either the standard power grid or a battery or solar cells, which allows them to be deployed in remote locations with minimal maintenance. Their low cost and maintenance requirements allows utilities to deploy a network of sensors, as an inexpensive way to monitor entire wastewater streams to quickly detect and locate any improper concentration of contaminants. Utilities, agencies, and industries may benefit from the timely and localized detection of contamination before pollutants are diluted by mixing with other streams so that appropriate action can be taken. In addition, the faster response time which this network of sensors permits will serve as a deterrent to waste dumping by companies because they will know that they will be quickly caught and prosecuted. Alternatively, these same sensors could be useful to industry as a way of preventing accidental discharge.

In addition to water monitoring for the specific chemicals to which the yeast have been genetically modified to respond, the present disclosure may be used for a range of other applications as part of a water monitoring or water treatment system. Notably, many of these other examples do not require the biological organisms to have been genetically modified. In addition to detecting the chemicals of interest, the yeast themselves can serve as sensors for acute toxicity, as detected by a drop in growth rate or culture density. Carbon dioxide can also be measured as emitted by the yeast; a drop in CO₂ may indicate a lower population of yeast. Furthermore, the yeast cultures may be additionally genetically modified to respond to other changes in their environment, including reporters for colorimetric signals, or for responses to changes in pH.

The present disclosure may also be configured to monitor eutrophication, or the presence of additional nutrients to the microorganisms. Microorganisms can also detect nutrients such as phosphate or nitrogen when the concentrated media has no or very low limits of the nutrient to be detected. In addition to yeast, salt-water bacteria may be used in this example, particularly in measuring the presence of nutrients in ocean water systems, such as in monitoring aquaculture. The growth rate of the microorganisms in the culture will then be limited by the amount of the nutrient, keeping the growth rate of the culture to a minimum. The concentration of the nutrient in the water can be determined by the growth rate, as measured by changes in culture density. If the growth rate increases above a threshold, an alarm is triggered. Alternatively, the culture density can be compared to standards to provide a quantitative measure of the amount of nutrient in the culture.

Mutagenicity in water sources can also be detected. Microorganisms grown for a period of months will accumulate mutations at a rate determined by the mutagenic activity of the water they are exposed to. Mutations can be assayed by performing whole genome sequencing or other methods on the cultures and comparing the rate of mutation to control cultures grown in controlled water conditions.

Other applications include ecosystem monitoring (e.g., researchers could study contaminant distribution throughout an ecosystem by deploying a network of sensors to monitor different locations in a watershed) and monitoring of long-term waste storage locations. Standard chemical testing may also be used in the detector device, where the water to be tested may be continuously or periodically mixed with a chemical test agent in a reaction chamber, and the sensor loop will assay the mixture for the result of the chemical test.

Although certain example methods and apparatus have been described herein, the scope of coverage of this patent is not limited thereto. On the contrary, this patent covers all methods, apparatus, and articles of manufacture fairly falling within the scope of the appended claims either literally or under the doctrine of equivalents.

What is claimed is:

1. A method of creating and maintaining a continuous culture biosensor system, the method comprising:
contacting an aqueous culture of a yeast capable of producing a fluorescent chemical and of altering a growth rate in response to an in situ threshold level of a specific contaminant in a chamber with an aqueous solution or continuous stream that may contain the specific contaminant;
providing nutrients to the yeast to sustain the yeast viability;
calculating the growth rate of the yeast by periodically measuring at least one of a relative density or a population count of the yeast and comparing the results to past ones;
transmitting data representative of the growth rate to a processor; and
determining the presence or lack of the specific contaminant by analyzing the growth rate to predetermined expected growth rate or measuring the presence of the fluorescent chemical that was produced by the yeast in response to the yeast coming into contact with the threshold level of the specific contaminant by use of a photodetector.

2. A method as recited in claim 1, wherein the contacting is aided by a pump.

3. A method as recited in claim 1, further comprising measuring at least one of the relative density or the population count of the yeast by use of the photodetector.

4. A method as recited in claim 1, further comprising measuring at least one of the relative density or the population count of the yeast by use of a CO₂ detector.

5. A method as recited in claim 1, further comprising preventing the introduction of a foreign biological organism from interrupting the growth or response of the continuous culture system, by use of at least one of a sanitizer, a filter, a settler, or a bacterial toxin.

6. A method as recited in claim 1, wherein the specific contaminant is at least one of estrogen, copper, arsenic, cobalt, zinc, cadmium, mercury, chromate, nickel, other metals, pharmaceuticals, endrocrine disruptors, fertilizers, pesticides, cleaning agents, industrial wastes, fracking chemicals, or petrochemicals.

7. A method as recited in claim 1, further comprising adding nutrients to stimulate growth of the yeast in response to a low measurement of the relative density or a population count of the yeast.

8. A method as recited in claim 1, wherein the specific contaminant is at least one of a phosphate or nitrogen compound.

9. A method as recited in claim 1, further comprising:
periodically measuring the genetic variability of the yeast; and
assessing the amount of genetic change by comparing to those seen in a control sample.

10. A method as recited in claim 9, wherein the genetic variability is measured via whole genome sequencing.

11. A method as recited in claim 9, further comprising measuring the concentration of yeast in the chamber by use of the photodetector.

12. A method as recited in claim 9, further comprising measuring the concentration of yeast in the chamber by use of a CO₂ detector.

13. A method as recited in claim 1, wherein the yeast is a genetically engineered yeast.
